(19) United States
(12) Patent Application Publication
(10) Pub. No.: US 2024/0149247 A1
(43) Pub. Date: May 9, 2024

(54) PHOTOCATALYTIC CONVERSION OF CARBON DIOXIDE AND WATER INTO HYDROCARBONS

(71) Applicant: University of Hull, Kingston upon Hull (GB)

(72) Inventor: Xuebin Ke, Kingston upon of Hull (GB)

(73) Assignee: University of Hull, Kingston upon Hull (GB)

(21) Appl. No.: 17/773,363

(22) PCT Filed: Oct. 29, 2020
(86) PCT No.: PCT/GB2020/052729

§ 371 Date: Apr. 29, 2022

(30) Foreign Application Priority Data
Oct. 29, 2019 (GB) 1915690.0

(51) Int. Cl. B01J 21/18; B01J 23/72; B01J 23/745; B01J 23/755; B01J 35/39; B01J 35/45; B01J 35/58; B01J 37/02; B01J 37/16; C07C 29/156; B82Y 30/00; B82Y 40/00

(52) U.S. Cl. CPC B01J 21/185; B01J 23/72; B01J 23/745; B01J 23/755; B01J 35/39; B01J 35/45; B01J 35/58; B01J 37/0219; B01J 37/0221; B01J 37/16; C07C 29/156; B82Y 30/00; B82Y 40/00

(57) ABSTRACT

The present invention relates to photocatalytic materials for use in the conversion of CO₂ to non-CO₂ carbon containing products. The photocatalytic materials comprise a metal nanofiber and a carbon-based nanostructure bound to the surface of the metal nanofiber. Methods for preparing such materials are described, as well as their use in the conversion of CO₂ to non-CO₂ carbon containing products. For example, the photocatalytic materials of the invention may be used to convert CO₂ to methanol and/or ethanol with high conversion rates.

This invention relates to photocatalytic materials that comprise a metal nanofiber and a plurality of carbon-based nanostructures bound to the surface of the metal nanofiber. Methods for preparing such materials are described, as well as their use in the conversion of CO₂ to non-CO₂ carbon containing products.

### BACKGROUND

Direct conversion of CO₂ into useful products is a crucial technique in situations where abundant and concentrated CO₂ is present, such as in the flue gas of power plants. Absorption of CO₂ with amines has demonstrated significant potential for such conversions, but large amounts of absorbents are consumed, which impede its extensive application in industry (E. S. Sanz-Pérez, C. R. Murdock, S. A. Didas, C. W. Jones, Direct capture of CO₂ from ambient air, Chem. Rev. 2016, 116, 11840-11876). Electrochemical conversion of CO₂ can directly convert CO₂ into valuable chemicals or fuels but this requires additional energy input (X. Chang, T. Wang, J. Gong, CO₂ photo-reduction: insights into CO₂ activation and reaction on surfaces of photocatalysts, Energy Environ. Sci. 2016, 9, 2177-2196). Photocatalytic conversion of CO₂ to hydrocarbon fuels or valuable chemicals represents a promising solution to the expense or energy requirements of these existing technologies.

Existing photocatalytic materials for the conversion of CO₂ into hydrocarbon products suffer from low absorption of light energy and low activating capability to drive the reactions to valuable products. Even during natural photosynthesis reactions in a rapidly growing tree, for example, a photosynthetic efficiency for conversion to hydrocarbon products is only around 1% (H. Michel, The nonsense of biofuels, Angew. Chem. Int. Ed. 2012, 51, 2516-2518).

U.S. patent application Ser. No. 13/896,987 describes a photocatalytic CO₂ reduction system for converting CO₂ into methane and water. The system employs photoactive materials such as plasmonic metal nanoparticles and photocatalytic capped colloidal nanocrystals (PCCN). These PCCN materials, which are semiconductor based, are deposited between the plasmonic nanoparticles to form a photocatalyst for redox reactions.

U.S. patent application Ser. No. 13/280,401 describes nanostructured arrays having a metal catalyst being irradiated with light to initiate artificial photosynthetic reactions resulting in the formation of carbon-containing molecules. The nanostructured arrays are formed from metals or layers of metals which may be used to enhance the irradiated light to dissociate molecules through single or multiple photon processes. Systems comprising nanostructured arrays were shown to produce between 2 ml/(gh) and 105 ml/(gh) of hydrocarbon products.

“Toward Solar Fuels: Photocatalytic Conversion of Carbon Dioxide to Hydrocarbons; Somnath Roy, Oomman Varghese, Maggie Paulose, and Craig Grimes, ACS Nano, 2010, 4(3), 1259-1278” reviews several metal oxide and semiconductor-based materials developed for converting CO₂ into hydrocarbon products. The review highlights the significant breakthrough in the photocatalytic reduction of gas phase CO₂ by solar radiation when using nitrogen doped TiO₂ nanotube arrays. These materials are quoted as having hydrocarbon production rates of up to 200 ppm cm⁻² h⁻¹.

Carbon nanotubes (CNTs) have been extensively studied and chemical vapour deposition (CVD) has been employed to produce CNTs (Synthesis of carbon nanotubes by catalytic chemical vapour deposition: A review on carbon sources, catalysts and substrates, Mater. Sci. Semicond. Process. 2016, 41, 67-82). Usually the transition metal nanoparticles, including Fe, Co, and Ni, are employed as catalysts in CVD synthesis of CNTs. The length of CNTs is from about 200 nm to 1 cm and the diameter is from several nanometers to tens of nanometers.

### BRIEF SUMMARY OF THE DISCLOSURE

In a first aspect, the invention provides a photocatalytic material comprising:
a metal nanofiber substrate; and
a carbon-based nano-structure, wherein the carbon-based nano-structure is bound to the surface of the metal nanofiber substrate.

In an embodiment, the photocatalytic material comprises a plurality of metal nanofiber substrates. Having a plurality of metal nanofiber substrates results in a high total surface area. For example, the total surface area of the metal nanofiber substrate may be above 100 m²/g. The surface area of the metal nanofiber substrate can be tuned according to the desired properties of the photocatalytic material. In contrast, conventional metal particles on substrates for photocatalysts have total surface area below 1 m²/g.

In an embodiment, the metal is one or more metals that permit surface plasmon resonance effects upon exposure to visible, ultraviolet, or infra-red wavelengths of electromagnetic radiation.

In an embodiment, the metal is selected from the group consisting of: Cu, Ni, Fe, Co, Ag, Pt, Mo, Au and a combination thereof. In an embodiment, the metal is selected from the group consisting of: Cu, Ni, Fe, Co, Ag, Pt, Mo and Au. In an embodiment, the metal is selected from the group consisting of: Cu, Ni, Fe, Co, Ag, Au and a combination thereof. In an embodiment, the metal is selected from the group consisting of: Cu, Ni, Fe, Co, Ag and Au.

In an embodiment, the term nanofiber includes metal substrates each having a high aspect ratio, i.e. length to diameter. In an embodiment, the length of the nanofiber is at least 50 times the diameter of the nanofiber. Optionally, the length of the nanofiber is at least 100 times the diameter of the nanofiber. Further optionally, the length of the nanofiber is at least 1000 times the diameter of the nanofiber.

In an embodiment, the metal nanofiber has a diameter in the range from 5 nm to 100 nm. Optionally, each metal nanofiber of the plurality of metal nanofibers has a diameter in the range from 10 nm to 80 nm. Further optionally, each metal nanofiber of the plurality of metal nanofibers has a diameter in the range from 20 nm to 70 nm. Further optionally, each metal nanofiber of the plurality of metal nanofibers has a diameter in the range from 30 nm to 60 nm. Further optionally, each metal nanofiber of the plurality of metal nanofibers has a diameter of approximately 50 nm.

In an embodiment, the metal nanofiber has a length in the range from 0.5 μm to 30 μm. Optionally, the metal nanofiber has a length in the range from 1 μm to 25 μm. Further optionally, the metal nanofiber has a length in the range from 5 μm to 20 μm. Still further optionally, the metal nanofiber has a length of approximately 10 μm.

The diameter and/or length of the metal nanofibers may be tailored to maximise the surface area from which the carbon-based nano-structure can be grown.

In an embodiment, the photocatalytic material comprises a plurality of carbon-based nano-structures. In an embodiment, the carbon-based nano-structure comprises one or more carbon-based structures selected from the group consisting of: a carbon-nanotube and graphene.

In an embodiment, the photocatalytic material comprises a metal nanofiber, wherein one or more carbon-nanotubes are bound to the surface of the metal nanofiber. In an embodiment, the material comprises a metal nanofiber, wherein one or more graphene sheets are bound to the surface of the metal nanofiber. In an embodiment, the photocatalytic material comprises a metal nanofiber, wherein one or more carbon-nanotubes and one or more graphene sheets are bound to the surface of the metal nanofiber.

The following embodiments relate to photocatalytic materials wherein the one or more carbon-based nanostructures are carbon nanotubes. In an embodiment each carbon nanotube has a diameter of less than 50 nm. Optionally, each carbon nanotube has a diameter in the range from 1 nm to 30 nm. Further optionally, each carbon nanotube has a diameter in the range from 1 nm to 25 nm, for example, from 1 nm to 10 nm or from 10 nm to 25 nm. Still further optionally, each carbon nanotube has a diameter in the range from 15 nm to 25 nm.

In an embodiment, the carbon nanotube is a multi-wall carbon nanotube (MWCNT). In an embodiment each MWCNT has a diameter of less than 50 nm. Optionally, each MWCNT has a diameter in the range from 1 nm to 30 nm. Further optionally, each MWCNT has a diameter in the range from 1 nm to 25 nm, for example, from 1 nm to 10 nm or from 10 nm to 25 nm. Still further optionally, each MWCNT has a diameter in the range from 15 nm to 25 nm.

The following embodiments relate to photocatalytic materials wherein the one or more carbon-based nanostructures are carbon nanotubes. In an embodiment each carbon nanotube has a length of less than 10 μm. Optionally, each carbon nanotube has a length in the range of 1 μm to 10 μm. Further optionally, each carbon nanotube has a length in the range of 2 μm to 8 μm. Still further optionally, each carbon nanotube has a length in the range of 4 μm to 6 μm.

In an embodiment, the carbon nanotube is a multi-wall carbon nanotube (MWCNT). In an embodiment each MWCNT has a length of less than 10 am. Optionally, each MWCNT has a length in the range of 1 μm to 10 μm. Further optionally, each MWCNT has a length in the range of 2 μm to 8 μm. Still further optionally, each MWCNT has a length in the range of 4 μm to 6 μm.

In embodiments where the carbon-based nanostructures are one or more graphene sheets/flakes. The growth of monolayer graphene has tuneable grain size ranging from 200 nm to 1 μm.

In an embodiment, the photocatalytic material comprises substantially more metal nanofiber material than carbon-based structure material, by weight. For example, the photocatalytic material comprises up to 40% carbon-based structure material by weight, up to 30% carbon-based structure material by weight, or up to 20% carbon-based structure material by weight. In some embodiments, the photocatalytic material comprises up to 10% carbon-based structure material by weight, e.g. 6% carbon-based structure material by weight.

The percentage composition of carbon-based nanostructure material may be selected to maximise the efficiency of the conversion of CO₂ to hydrocarbon products. For example, the amount of carbon-based nanostructure material may be selected as a balance of maximising the number of active sites on the carbon-based nanostructure material against the ability of light to reach the metal substrate material for initiating surface plasmon resonance effects. In some embodiments, the photocatalytic material comprises 10% to 20% carbon-based structure material by weight.

In an embodiment, the photocatalytic material comprises a plurality of metal nanofibers, wherein one or more carbon-nanotubes are bound to the surface of the plurality of metal nanofibers. In an embodiment, the material comprises a plurality of metal nanofibers, wherein one or more graphene sheets are bound to the surface of the plurality of metal nanofibers. In an embodiment, the photocatalytic material comprises a plurality of metal nanofibers, wherein one or more carbon-nanotubes and one or more graphene sheets are bound to the surface of the plurality of metal nanofibers.

In an embodiment, the photocatalytic material comprises a plurality of metal nanofibers, wherein the plurality of metal nanofibers have a plurality of carbon nanotubes extending from their surface, wherein the metal is selected from copper, nickel, iron, or cobalt.

The term ‘bound’ includes chemical bonding, e.g. covalent bonding, ionic bonding, co-ordinate covalent bonding, and van-der Waals bonding. In an embodiment, the one or more carbon-based nanostructures are covalently bonded to the surface of the metal nanofiber. Not wishing to be bound by theory, the one or more carbon-based nanostructures may be covalently bonded to the surface of the metal nanofiber via surface-bound oxygen.

Again, without wishing to be bound by theory, it is thought that the metal nanofibers act as an absorber of visible, UV, and IR radiation to generate ‘hot’ electrons. The catalytic material includes a heterojunction between the metal nanofiber substrate and the carbon-based nanostructures. The heterojunction arises due to the carbon nanostructures being grown from the metal nanofibers rather than being merely associated with or deposited onto the metal nanofiber.

The heterojunction permits ‘hot’ electrons to transfer between the metal nanofibers through the carbon nanostructures rapidly. The transfer of the electrons to the carbon nanostructure component of the material permits catalytic reduction of carbon dioxide to occur on the metal nanofiber.

In a second aspect, the invention provides a method of producing a photocatalytic material, the method comprising:
providing a metal nanofiber substrate; and
growing from the surface of the metal nanofiber substrate, a carbon-based nanostructure.

In some embodiments, the step of growing the carbon-based nanostructure from the surface of the metal nanofiber substrate involves exposing the metal nanofiber substrate to a carbon source at high temperature.

In an embodiment, the step of growing the carbon-based nanostructures from the surface of the metal nanofiber substrate involves exposing the metal nanofiber substrate to a carbon source at high temperature in the absence of oxygen (to avoid oxidation). Optionally, the step of growing the carbon-based nanostructures from the surface of the metal nanofiber substrate involves exposing the metal oxide nanofiber substrate to a carbon source at high temperature in the presence of an inert carrier gas, for example nitrogen.

In preferred embodiments, the metal nanofiber substrate is exposed to a carbon source under conventional chemical vapour deposition (CVD) techniques. Chemical vapour deposition can be achieved by reacting the metal nanofiber substrate with a carbon source in a chemical vapour deposition reactor.

In an embodiment, the step of providing a metal nanofiber substrate comprises forming the metal nanofiber substrate. The step of forming the metal nanofiber substrate may itself be a multi-step process. For example, the metal nanofiber substrate may be produced by first forming a metal oxide nanofiber substrate. The method may then comprise the additional step of converting (i.e. reducing) the metal oxide nanofiber substrate to a metal nanofiber substrate.

Therefore, in an embodiment, the method of producing a photocatalytic material comprises the steps of:
providing a metal oxide nanofiber substrate;
converting the metal oxide nanofiber substrate to a metal nanofiber substrate; and
growing from the surface of the metal nanofiber substrate, a carbon-based nanostructure.

In an embodiment, the step of providing a metal oxide nanofiber substrate comprises forming the metal oxide nanofiber substrate. The metal oxide nanofiber substrate can be manufactured by conventional means, for example, by combining a metal (e.g. Cu, Ni, Fe, Co, Ag or Au) salt with KOH, followed by addition of NH₃ and filtering. As an example, the process described in the experimental section of “Simple Template-Free Solution Route for the Controlled Synthesis of Cu(OH)₂ and CuO Nanostructures; Conghua Lu, Limin Qi, Jinhu Yang, Dayong Zhang, Nianzu Wu, and Jiming Ma, J. Phys. Chem. B 2004, 108 (46), 17825-17831” may be used to prepare CuO nanofibers.

In an embodiment, the step of converting a metal oxide nanofiber substrate to a metal nanofiber substrate is performed in conjunction with the step of growing a carbon-based nanostructure from the surface of the metal oxide nanofiber substrate. Simultaneous conversion of a metal oxide nanofiber to a metal nanofiber and growing of the carbon-based nanostructures enables the formation of stable metal nanofibers, i.e. the simultaneous reduction of metal oxide to metal and growth of carbon-based structures prevents multiple metal nanofiber substrates from aggregating into a single metal structure.

In some embodiments, the step of growing the carbon-based nanostructure from the surface of the metal oxide nanofiber substrate also performs the function of reducing the metal oxide nanofiber substrate to a metal nanofiber substrate. For example, the metal nanofiber substrate may be formed by exposing the metal oxide nanofiber substrate to a carbon source at high temperature.

In an embodiment, the step of growing the carbon-based nanostructures from the surface of the metal oxide nanofiber substrate involves exposing the metal oxide nanofiber substrate to a carbon source at high temperature in the absence of oxygen (to avoid oxidation). Optionally, the step of growing the carbon-based nanostructures from the surface of the metal oxide nanofiber substrate involves exposing the metal oxide nanofiber substrate to a carbon source at high temperature in the presence of an inert carrier gas, for example nitrogen.

In preferred embodiments, the metal oxide nanofiber substrate is exposed to a carbon source under conventional chemical vapour deposition (CVD) techniques. The CVD technique may perform the function of reducing the metal oxide nanofiber substrate and of growing the carbon-based structure from the surface of the metal nanofiber substrate. Chemical vapour deposition can be achieved by reacting the metal oxide nanofiber substrate with a carbon source in a chemical vapour deposition reactor.

In an embodiment, the carbon source is selected from the group consisting of: cellulose, high-density polyethylene (HDPE), polypropylene and polyethylene terephthalate (PET) plastics. In an embodiment, the carbon source is cellulose.

In an embodiment, the carbon source is pre-heated before being exposed to the metal oxide nanofiber substrate. Optionally, the carbon source is pre-heated to a temperature of from 300° C. to 800° C. Further optionally, the carbon source is pre-heated to a temperature of from 400° C. to 700° C. Still further optionally, the carbon source is pre-heated to a temperature of from 400° C. to 600° C., for example about 500° C. The pre-heating step assists in ensuring pyrolysis of the carbon source.

In an embodiment, the carbon source is exposed to the metal or metal oxide nanofiber substrate by introducing to the metal or metal oxide nanofiber substrate at a feed rate of from 1 mL/min to 100 mL/min. Optionally, the feed rate is from 1 mL/min to 50 mL/min. Further optionally, the feed rate is from 1 mL/min to 25 mL/min. Still further optionally, the feed rate is about 10 mL/min.

In an embodiment, the metal or metal oxide nanofiber substrate is pre-heated before being exposed to the carbon source. Optionally, the metal or metal oxide nanofiber substrate is pre-heated to a temperature of from 200° C. to 400° C. Further optionally, the metal or metal oxide nanofiber substrate is pre-heated to a temperature of from 250° C. to 350° C. The pre-heating step assists in ensuring pyrolysis of the carbon source.

In an embodiment, the metal or metal oxide nanofiber substrate is heated to a temperature of from 600 to 900° C. whilst exposing to the carbon source. Optionally, the metal or metal oxide nanofiber substrate is heated to a temperature of from 700 to 800° C. whilst exposing to the carbon source. Further optionally, the metal or metal oxide nanofiber substrate is heated to a temperature of 750° C. whilst exposing to the carbon source.

In an embodiment, the step of forming the metal nanofiber substrate comprises forming a plurality of metal nanofibers. Alternatively, in embodiments where the metal nanofiber substrate is formed via a metal oxide nanofiber substrate, the step of forming the metal nanofiber substrate comprises forming a plurality of metal oxide nanofibers that are subsequently converted to metal nanofibers.

In an embodiment, the growing step comprises growing a plurality of carbon-based nanostructures.

In an embodiment, the method of producing the photocatalytic material comprises providing a plurality of metal nanofiber substrates and growing a plurality of carbon-based nanostructures.

In an embodiment, the method of producing the photocatalytic material comprises the steps of:
providing a plurality of metal oxide nanofibers; and
exposing the metal oxide nanofibers to a carbon source at high temperature, such that the plurality of metal oxide nanofibers are reduced to a plurality of metal nanofibers as the plurality of carbon nanotubes are grown from the surface of the plurality of metal oxide nanofibers; wherein the metal is selected from copper, nickel, iron, or cobalt.

In an embodiment, the method of producing the photocatalytic material comprises the steps of:
providing a plurality of copper oxide nanofibers; and
exposing the copper oxide nanofibers to a carbon source at high temperature, such that the plurality of copper oxide nanofibers are reduced to a plurality of copper nanofibers as the plurality of carbon nanotubes are grown from the surface of the plurality of copper nanofibers.

The photocatalytic material according to the first aspect may be produced by the method of the second aspect. Thus, any embodiment described above according to the first aspect may apply, where appropriate, to the second aspect of the invention. In addition, any embodiment described above according to the second aspect may apply, where appropriate, to the first aspect.

In a third aspect, the invention provides a method of converting CO₂ to at least one non-CO₂ carbon containing product, the method comprising:
exposing a photocatalytic material according to the first aspect to CO₂;
irradiating the photocatalytic material with a light source in the presence of the CO₂.

In an embodiment, the method of converting CO₂ to at least one non-CO₂ carbon containing product further comprises the step of suspending the photocatalytic material in a liquid. In an embodiment the liquid is water.

In an embodiment, the liquid for suspending the photocatalytic material further comprises additives to aid the photocatalytic reduction of CO₂ to at least one non-CO₂ carbon containing product. For example, the additive may be selected from an acid or a base. In embodiments, the additive is triethanolamine or isopropanol.

In embodiments, the step of exposing the photocatalytic material to CO₂ further comprises the step of purging the reaction vessel in which the reaction occurs with CO₂. This purging step may occur for a period of time prior to the irradiation step. The purging step may be performed for as long as necessary to substantially exclude oxygen from the reaction vessel. In some embodiments, the purging step is continued during the irradiation step.

In embodiments, the step of irradiating the photocatalytic material with a light source comprises using a light source that produces visible light, ultraviolet light, infra-red radiation or any combination of these. The light source may produce visible light, ultraviolet light, and infra-red radiation. In some embodiments, the light source may be the sun.

In embodiments, the step of irradiating the photocatalytic material with a light source is performed for any period of time sufficient to produce an amount of at least one non-CO₂ carbon containing product. In embodiments, the step of irradiating the photocatalytic material is performed from 1 to 24 hours.

In some embodiments, the step of irradiating the photocatalytic material is performed continuously.

In some embodiments, the step of irradiating the photocatalytic material is performed without any control of the temperature of the reaction vessel. For example, the step of irradiating the photocatalytic material may be performed under ambient temperature conditions.

In some embodiments, the photocatalytic material may be recovered and reused.

In some embodiments, the at least one non-CO₂ carbon containing product is a hydrocarbon. In some embodiments, the at least one hydrocarbon product is methanol. In some embodiments, the at least one hydrocarbon product is ethanol. In some embodiments, the at least one hydrocarbon product is a mixture of methanol and ethanol.

Without wishing to be bound by theory, it is thought that the photocatalytic conversion of CO₂ to non-CO₂ carbon containing products occurs via mechanisms discussed in “Electrochemical CO₂ reduction: Electrocatalyst, reaction mechanism, and process engineering; Qi Lu, Feng Jiao, Nano Energy 29 (2016), 439-456” and “Photochemical and Photoelectrochemical Reduction of CO₂; Bupendra Karma et al. Annu. Rev. Phys. Chem. 2012 63:541-69”:

CO₂+2H⁺+2e⁻→HCOOH (eq. 1)

CO₂+2H⁺+2e⁻→CO+H₂O (eq. 2)

CO₂+4H⁺+4e⁻→C+2H₂O (eq. 3)

CO₂+4H⁺+4e⁻→HCHO+H₂O (eq. 4)

CO₂+6H⁺+6e⁻→CH₃OH+H₂O (eq. 5)

CO₂+8H⁺+8e⁻→CH₄+2H₂O (eq. 6)

Without wishing to be bound by theory, it is thought that the photocatalytic conversion of CO₂ to ethanol occurs via a further reaction of any of the initial products in equations 1-6.

The photocatalytic material according to the first aspect or the photocatalytic material produced by the second aspect may be used in the method of the third aspect. Thus, any embodiment described above according to the material of the first aspect may apply, where appropriate, to the third aspects of the invention. In addition, any embodiment described above according to the third aspect may apply, where appropriate, to the first or second aspects.

### BRIEF DESCRIPTION OF THE DRAWINGS

Embodiments of the invention are further described hereinafter with reference to the accompanying drawings, in which:

FIG. 1 is an SEM image of copper oxide nanofibers produced according to the methods described herein.

FIG. 2A is an EDS spectra for a photocatalytic material according to the present invention. The EDS spectra demonstrates a carbon:copper ratio of 30:70.

FIG. 2B is an EDS spectra for a photocatalytic material according to the present invention. The EDS spectra demonstrates a carbon:copper ratio of 10:90.

FIG. 3 is the recycling performance of photocatalysts produced according to the methods described herein. The left hand bar of each pair of bars is CH₃OH and the right hand bar of each pair of bars is C₂H₅OH.

FIG. 4A is an TEM image of CNTs based on nickel nanofibers produced according to the methods described herein. FIG. 4B is an SEM image of CNTs based on nickel nanofibers produced according to the methods described herein.

FIG. 5A is an TEM image of CNTs based on iron nanofibers produced according to the methods described herein. FIG. 5B is an SEM image of CNTs based on iron nanofibers produced according to the methods described herein.

FIG. 6 is an SEM image of nickel oxide nanofibers produced according to the methods described herein.

FIG. 7 is an SEM image of iron oxide nanofibers produced according to the methods described herein.

FIG. 8 is an EDS map that illustrates mapping of Fe (top right) and mapping of C (bottom right) relative to the SEM image of the material (left).

### DETAILED DESCRIPTION

Throughout this specification, whenever a specific value is quoted for a temperature, pressure or time, the temperature, pressure or time quoted is approximate rather than the precise temperature, amount of pressure or amount of time. Nevertheless, the disclosure includes the precise value of any such variables which are approximately that value.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

### Example 1—Materials Fabrication: Based on Cu Nanofibers

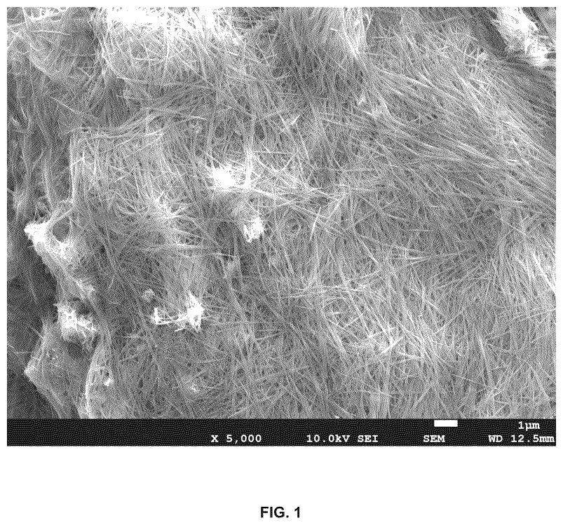

40 mL of 1M KOH solution was first dropped into 100 mL of 0.1 M CuSO₄ solution under stirring at ambient temperature. Then 15 mL of ammonia solution (12.5 wt %) was added. After stirring for 3 min, the mixed solution was kept static for 12-16 h. The resultant precipitate was filtered off, washed with deionized water, and dried for 12 h. The resulting CuO nanofibers can be seen in the SEM image in FIG. 1.

A two-stage tubular furnace was set as a CVD reactor and N₂ were used as the carrier gas. The substrates of CuO nanofibers were placed in the middle of the quartz tube on a quartz tray. Cellulose were used as carbon source and was heated to 500° C. with the feed rate of 10 mL/min. The substrates were preheated to 250° C. for 30 min and then increased to 750° C. maintaining for 1 h to fabricate carbon nanotubes.

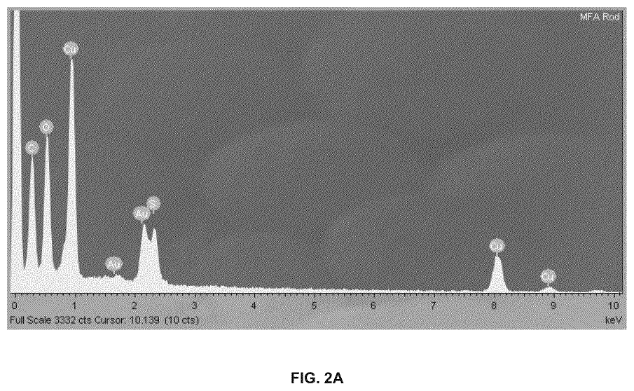

The energy-dispersive X-ray spectroscopy (EDS) data shown in FIGS. 2A and 2B demonstrate that the growth of carbon nanotubes on the CuO nanofibers results in the photocatalytic material having a ratio of carbon:copper from 30:70 to 10:90.

### Example 2: Materials Fabrication: Based on Ni Nanofibers

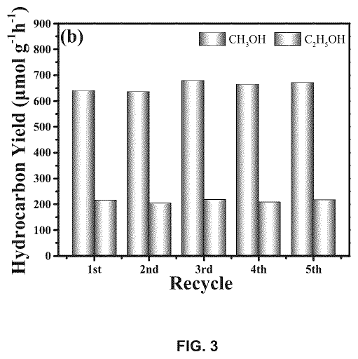

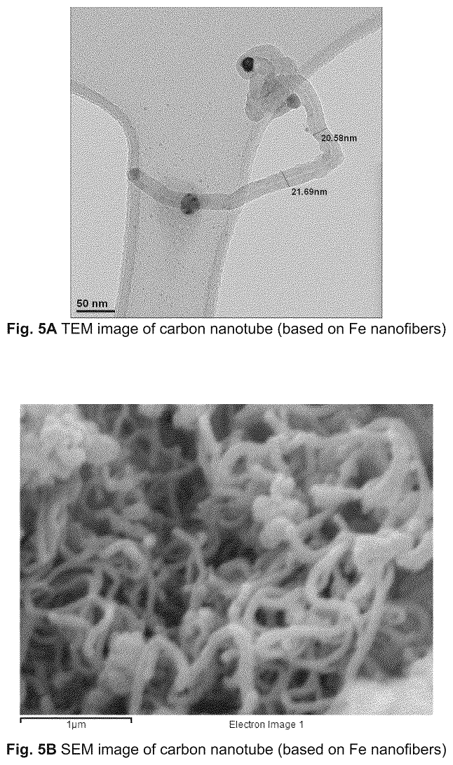

100 ml of 0.1 M NiCl₂·6H₂O solution mixed with 0.630 g (4.5 mmol) of Na₂C₂O₄. Then 150 ml ethylene glycol and 4.5 g PEG were added. The solution was transferred into a Teflon-lined stainless steel autoclave, sealed and heated at 220° C. for 12 h. The resultant precipitate was filtered off, washed with deionized water, and dried for 12 h. The resulting NiO nanofibers can be seen in the SEM image in FIG. 6. The CVD process is similar to Example 1. SEM and TEM images of these materials are provided in FIGS. 4A and 4B.

### Example 3: Materials Fabrication: Based on Fe Nanofibers

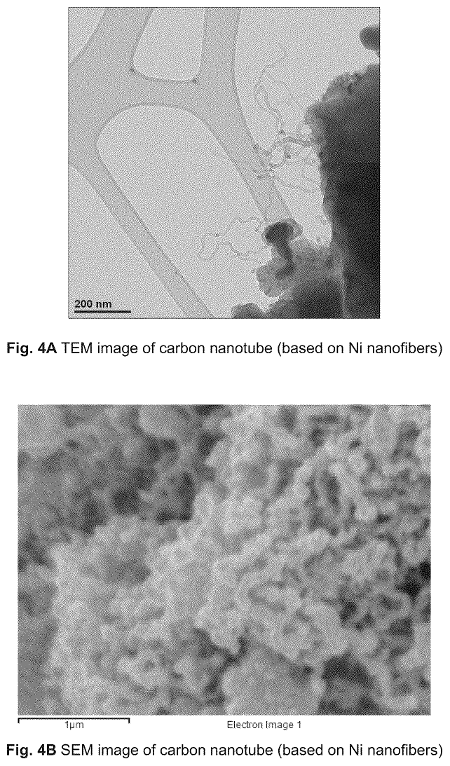

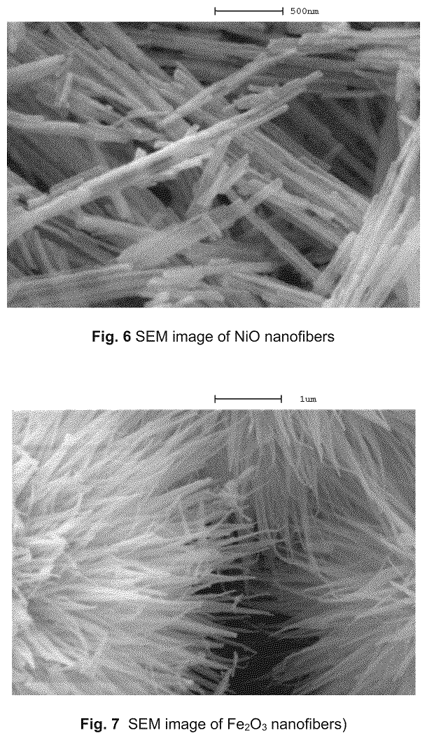

0.15 M FeCl₃ aqueous solution was mixed with isopropanol, to which 3 mmol nitrilotriacetic acid (NTA) was added. After thorough stirring, the mixture was transferred into a Teflon lined autoclave and hydrothermally treated at 180° C. for 24 h. The resultant precipitate was filtered off, washed with deionized water, and dried for 12 h. The resulting Fe₂O₃ nanofibers can be seen in the SEM image in FIG. 7. The CVD process is similar to Example 1. SEM and TEM images of these materials are provided in FIGS. 5A and 5B.

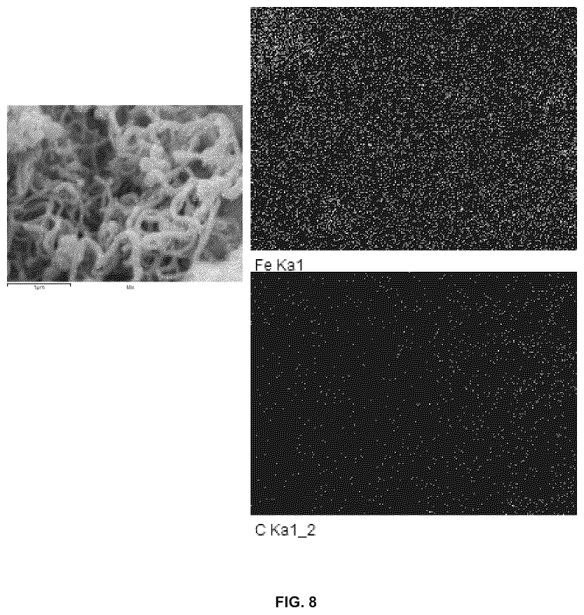

An EDS map that illustrates mapping of Fe and mapping of C relative to the SEM image of the material is provided in FIG. 8. From this Figure it can be seen that both carbon and iron are well distributed in the final material and also that the tube-like structures in the SEM image are carbon-based.

### Example 4: CO₂ Conversion Performance

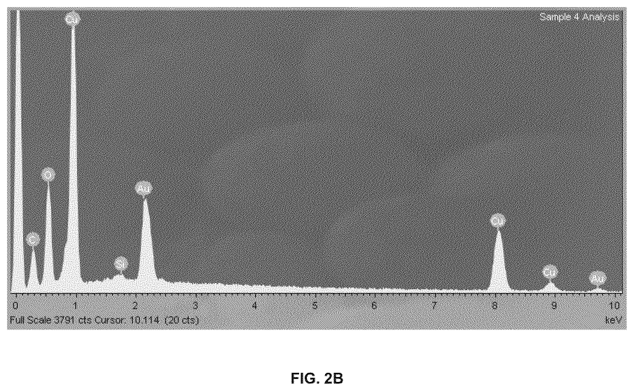

Photocatalytic CO₂ reduction was performed in a 50 mL reactor under ambient conditions. The photocatalytic material obtained in Example 1 (20 mg), triethanolamine (TEOA, 2 mL) and H₂O (20 mL) was added into the reactor, which was purged with CO₂ for 30 min. The reaction was carried out under light-irradiation by using a 300 W Xe lamp. The product was analyzed by gas chromatography. The durability of photocatalyst was assessed in a five-run recycling test under visible-light irradiation for 1 hour per cycle. FIG. 3 shows that no significant deactivation was observed between irradiation cycles. The CH₃OH and C₂H₅OH production rate was stable at 600-700 and 200 μmol g⁻¹ h⁻¹, respectively.

1. A photocatalytic material comprising:
a metal nanofiber substrate; and
a carbon-based nano-structure, wherein the carbon-based nano-structure is chemically bonded to the surface of the metal nanofiber substrate, and wherein the carbon-based nano-structure comprises a carbon nanotube.

2. The photocatalytic material according to claim 1, comprising a plurality of metal nanofiber substrates and a plurality of carbon-based nano-structures.

3. The photocatalytic material of claim 1, wherein the metal nanofiber substrate comprises a metal selected from the group consisting of: Cu, Ni, Fe, Co, Ag, Pt, Mo, Au and a combination thereof.

4. The photocatalytic material of claim 1, wherein each metal nanofiber substrate has a length and a diameter having high aspect ratio; optionally wherein the length of each metal nanofiber is at least 50 times the diameter of the nanofiber.

5. The photocatalytic material of claim 1, wherein each metal nanofiber has a diameter in the range from 5 nm to 100 nm.

6. The photocatalytic material of claim 1, wherein each metal nanofiber has a length in the range from 0.5 μm to 30 μm.

7. (canceled)

8. (canceled)

9. (canceled)

10. The photocatalytic material of claim 1, wherein each carbon nanotube has a diameter of less than 50 nm and a length of less than 10 μm.

11. The photocatalytic material of claim 1, wherein the carbon nanotube is a multi-wall carbon nanotube (MWCNT).

12. (canceled)

13. The photocatalytic material of claim 1, wherein the photocatalytic material comprises substantially more metal nanofiber material than carbon-based structure material, by weight; optionally wherein the photocatalytic material comprises up to 40% carbon-based structure material by weight.

14. A method of producing a photocatalytic material, the method comprising:
providing a metal nanofiber substrate; and
growing from the surface of the metal nanofiber substrate, a carbon-based nanostructure,
wherein the carbon-based nano-structure comprises a carbon nanotube.

15. The method of claim 14, wherein the step of growing the carbon-based nanostructure from the surface of the metal nanofiber substrate involves exposing the metal nanofiber substrate to a carbon source at high temperature; optionally in the absence of oxygen; and further optionally in the presence of an inert carrier gas.

16. The method of claim 14, wherein the metal nanofiber substrate is exposed to a carbon source under conventional chemical vapour deposition (CVD) techniques.

17. The method of claim 14, wherein the method of producing the photocatalytic material comprises the steps of:
providing a metal oxide nanofiber substrate;
converting the metal oxide nanofiber substrate to the metal nanofiber substrate; and
growing from the surface of the metal nanofiber substrate, the carbon-based nanostructure.

18. The method of claim 17, wherein the step of providing the metal oxide nanofiber substrate comprises forming the metal oxide nanofiber substrate, wherein the step of converting the metal oxide nanofiber substrate to the metal nanofiber substrate is performed in conjunction with the step of growing the carbon-based nanostructure from the surface of the metal oxide nanofiber substrate, and wherein the metal oxide nanofiber substrate is exposed to a carbon source under conventional chemical vapour deposition (CVD) techniques to reduce the metal oxide nanofiber substrate and to grow the carbon-based structure from the surface of the metal nanofiber substrate.

19. (canceled)

20. (canceled)

21. The method of claim 15, or wherein the carbon source is selected from the group consisting of: cellulose, high-density polyethylene (HDPE), polypropylene and polyethylene terephthalate (PET) plastics.

22. The method of claim 14, wherein the step of providing a metal nanofiber substrate comprises providing a plurality of metal nanofibers.

23. A method of converting CO₂ to at least one non-CO₂ carbon containing product, the method comprising:
exposing a photocatalytic material according to claim 1 to CO₂;
irradiating the photocatalytic material with a light source in the presence of the CO₂.

24. The method of claim 23, further comprising the step of suspending the photocatalytic material in a liquid; optionally wherein the liquid is water, and wherein the liquid for suspending the photocatalytic material further comprises additives; optionally wherein the additive is selected from an acid or a base: further optionally wherein the additive is selected from triethanolamine and isopropanol.

25. (canceled)

26. The method of claim 23, wherein the step of irradiating the photocatalytic material with a light source comprises using a light source that produces visible light, ultraviolet light, infra-red radiation or any combination of these.

27. The method of claim 23, wherein the at least one non-CO₂ carbon containing product is a hydrocarbon; optionally wherein the at least one hydrocarbon product is methanol or ethanol; further optionally wherein the at least one hydrocarbon product is a mixture of methanol and ethanol.
